(19) United States
(12) Patent Application Publication
(10) Pub. No.: US 2006/0204458 A1
(43) Pub. Date: Sep. 14, 2006

(54) Anti-aging methods and composition

(76) Inventors: William D. Holloway JR., Carlsbad, CA (US); Michael A. Holloway, Escondido, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 11/302,967

(22) Filed: Dec. 13, 2005

Related U.S. Application Data
(63) Continuation-in-part of application No. 10/420,280, filed on Apr. 21, 2003.
Application No. 10/420,280 is a continuation-in-part of application No. 10/301,416, filed on Nov. 21, 2002.
Application No. 10/301,416 is a continuation-in-part of application No. 09/698,537, filed on Oct. 26, 2000, now Pat. No. 6,521,248.
(60) Provisional application No. 60/635,915, filed on Dec. 13, 2004.
(60) Provisional application No. 60/596,170, filed on Sep. 6, 2005.
(60) Provisional application No. 60/594,612, filed on Apr. 22, 2005.
(60) Provisional application No. 60/594,540, filed on Apr. 15, 2005.
(60) Provisional application No. 60/595,095, filed on Jun. 6, 2005.

(51) Int. Cl. A61K 8/29; A61K 8/27

(52) U.S. Cl. 424/59

(57) ABSTRACT

An anti-aging method and composition are disclosed. The composition is a micro-cluster water having anti-oxidant and free-radical quenching properties. The method involves contacting a cell or portion thereof in danger of free-radical or oxidant damage with the micro-water.

### CROSS-REFERENCE TO RELATED APPLICATIONS

This application claims priority to each of provisional application No. 60/635,915, filed Dec. 13, 2004 and No. 60/596,170, filed Sep. 6, 2005, and is a continuation-in-part of application Ser. No. 10/420,280, filed Apr. 21, 2003; which is a continuation-in-part of application Ser. No. 10/301,416 filed Nov. 21, 2002, which is a continuation-in-part of application Ser. No. 09/698,537, filed Oct. 26, 2000, now issued as U.S. Pat. No. 6,521,248, which claims priority to provisional application No. 60/161,546, filed Oct. 26, 1999.

This application is also related to provisional applications No. 60/594,612 filed Apr. 22, 2005 and No. 60/594,540, filed Apr. 15, 2005. Each of the above-identified applications is incorporated by reference in its entirety.

### BACKGROUND OF THE INVENTION

Aging is an inevitable biological process generally characterized by decline in physiological function that leads to morbidity and mortality. The aging process occurres gradually over a person's lifetime. During this gradual process the decline in physiological function is exemplified as a general decrease in physical and mental ability. Moreover there is a progressive decline in strength of the immune system, with decreased ability for the aging body to heal.

Aging involves death of cells or cell dysfunction due to production of free radicals, oxidative damage and energy depletion due to mitochondrial dysfunction. Harman (1988) linked senescence or death to the injurious effects of free radicals arising from the one-electron reduction of oxygen during metabolism. There has been an inverse relationship between auto-oxidation rate in different animal species and life expectancy in the same species (Cutler 1985; Sohal 1995). Mitochondria are the major source of oxygen radicals through the respiratory chain and are also deeply affected by reactive oxygen species (ROS), resulting in serious risks to their function. Mitochondrial dysfunction could result in defects in electron transport, oxidative phosphorylation and energy production resulting in cell damage and ultimately cell death.

Although the exact cause for these declining propensities is not known, it has been proposed that damage to one or more of; cell membranes, electron transfer, brain tissue and disruption to energy related metabolic pathways which is caused by free-radicals and oxidants plays a significant causative role. An increase in oxidative lesions in mitochondrial DNA is observed on older subjects as compared to younger. This increase is observed in numerous tissue and cell types including, brain, muscle, nerve and diaphragm. Importantly, these increases are a comparative along the continuum of aging.

Damage to the mitochondrial DNA is of particular importance because of their ubiquitous involvement in energy production. Likewise, mitochondria are a ubiquitous organelle with a function of paramount importance. Oxidative damage to the brain mitochondrial DNA has been linked to increased incidence of neurodegenerative diseases with aging. Oxidative damage to muscular mitochondria has been linked to increased lethargy. Toxicity by oxygen radicals has also been suggested as a major cause of cancer, heart disease and aging in general.

A marked increase in life span has occurred within human evolution over the past 60 million years. At the same time an enormous decrease in the age-specific cancer rate has occurred in humans. It is likely that a major factor in lengthening life span and decreasing age-specific cancer rates may have been the evolution of effective mechanisms against free radicals and other sources of oxidative damage. Increasing the plasma concentration of radical and oxidant quenching compositions has been proposed. Compositions such as uric acid, vitamins A, E and C have been extensively studied in this regard, with limited success. One property shared by the vitamins is their lack of water solubility. Their plasma concentration is limited because of this lack of solubility in (water) blood. Moreover, these all share the property of undergoing digestive degradation, further complicating efforts towards increasing plasma concentration. Further problems involve transport of these anti-oxidants into the cells, where the oxidative damage is problematic.

One important attribute of an anti-oxidant or a free-radical quenching agent is the ability to chemically react with the oxidant or free-radical, in a biologically non-destructive manner. Several of the biologically destructive oxidants and free-radicals involved include superoxide (O₂⁻), H₂O₂, hydroxyl radicals (.OH) and singlet oxygen (¹O₂). The need exists for an anti-oxidant and free-radical quenching composition having desired solubility and cell uptake properties. The present micro-cluster water provides these and other beneficial properties.

### BRIEF SUMMARY OF THE INVENTION

One aspect of this invention is directed to a method and composition for quenching free-radicals and oxidants in intracellular fluids.

Another aspect of this invention is directed to a method and composition for quenching free-radicals and oxidants in extra-cellular fluids.

Another aspect of this invention is directed to a method and composition for quenching free-radicals and oxidants in intercellular fluids.

A still further aspect of the present invention is directed to a method and composition for the delivery of cosmeceuticals.

A further aspect of the present invention is a micro-cluster liquid having anti-oxidant and free-radical quenching properties.

The micro-cluster liquid, such as micro-cluster water, of the present invention further provides at least one property selected from the group comprising, increased potential energy, enhanced bioavailability, transdermal migration and transdermal facilitator.

The term transdermal migration shall mean having the ability to migrate through or across the dermis. Whereas the term transdermal facilitator shall mean the property of facilitating the transdermal passage of another substance across the dermal membrane often coincident with its own migration.

The term cosmaceutical shall mean a cosmetic formulation which includes at least one nutritional and/or pharmaceutical agent. A cosmaceutical may, for example, incorporate titanium dioxide (as the physical sunblock) and creatine pyruvate (as the cellular repair pharmaceutical) in a micro-cluster water vehicle. This composition may further include permeation enhancers to further facilitate delivery of the pharmaceutical deep into the dermis. The anti-oxidant and anti-free radical properties of the present micro-cluster water further provide the un-expected result of increased cell-longevity, decreased DNA mutation rates, increased mitochondria cell membrane longevity and increased cellular membrane longevity, in general.

### BRIEF DESCRIPTION OF THE DRAWINGS

FIG. 1 is a bar graph comparing the uptake of creatine into cells and mitochondria.

FIG. 2 is a graph depicting the gel and bar graphs of creatine and normal cells response to UVA-induced mtDNA mutagenesis.

FIG. 3 is a graphic of the chronic oxidative stress cycle.

FIG. 4 is a graphic of the defective powerhouse model of cutaneous aging.

FIG. 5 is a graph comparing the number of mutations between cells grown in media made with micro-cluster water (Penta) or lab water (a.d.).

FIG. 6 is a set of bar graphs comparing the number of common deletion mutations between cells grown media made with micro-cluster water (Penta) or lab water (a.d.).

FIG. 7 is a set of bar graphs comparing the number of common deletion mutations between cells grown media made with micro-cluster water (Penta) or lab water (a.d.).

FIGS. 8a and 8b are graphs showing the differences between double distilled water (“DDW”) and micro-cluster water (Micro-cluster) regarding changes to intracellular pH under standard incubation conditions.

FIG. 9 is a bar graph representation of the intracellular pH change in macrophages under standard incubation conditions.

FIG. 10 is a bar graph of the differences in propensity for damage to cellular membranes under standard incubation conditions, where the incubation medium was made from DDW or Micro-cluster water.

FIG. 11 is a bar graph comparing the occurrence of common deletion mutations in mtDNA between UV exposed skin and un-exposed skin.

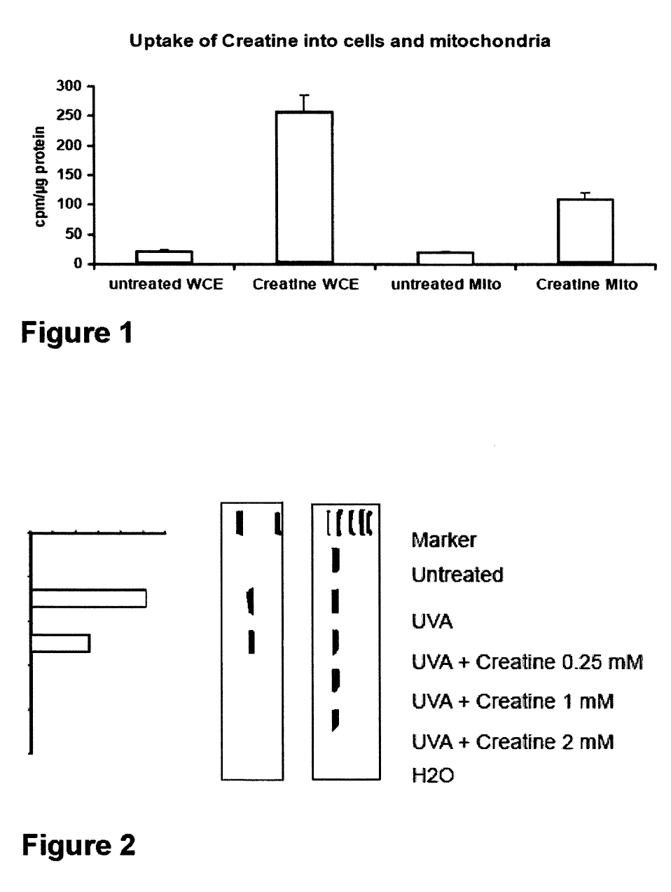

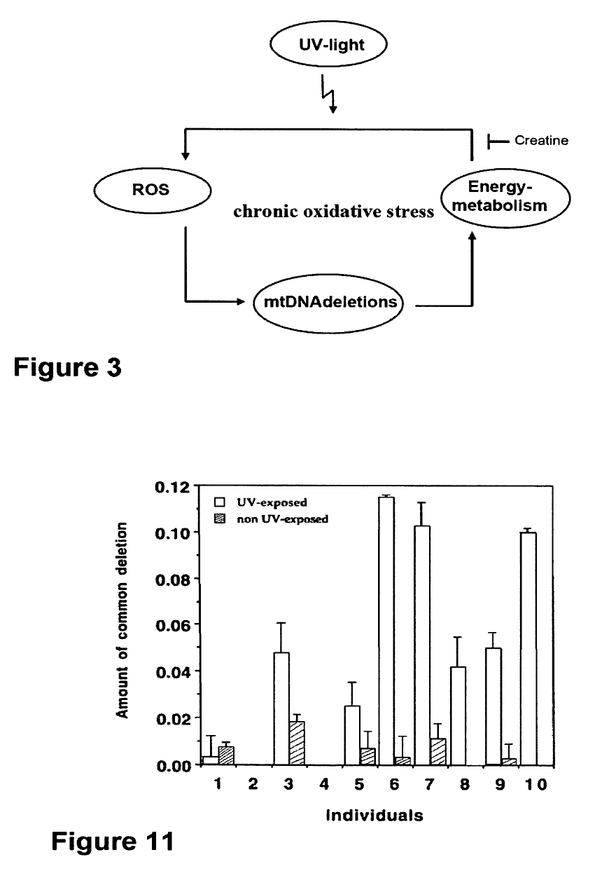

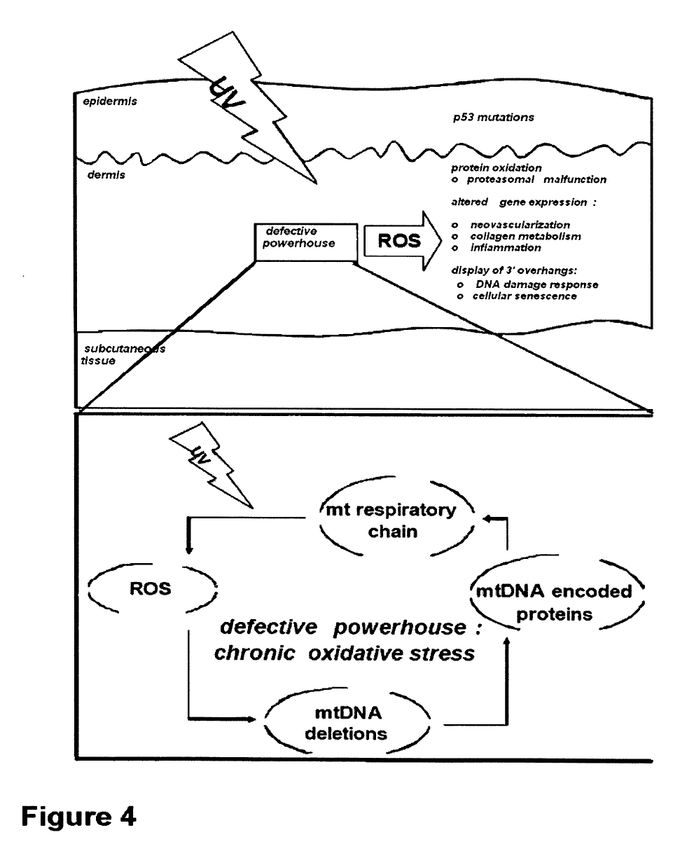

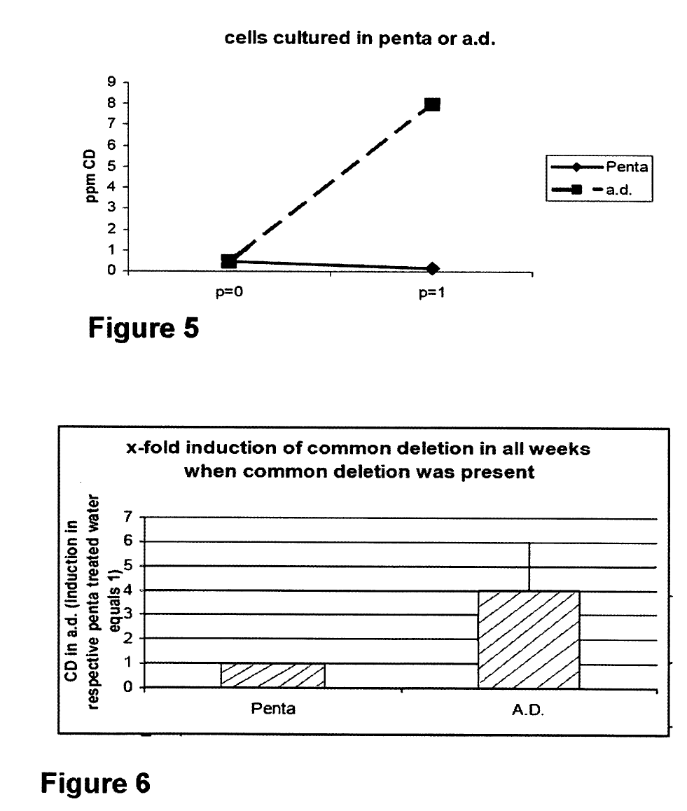

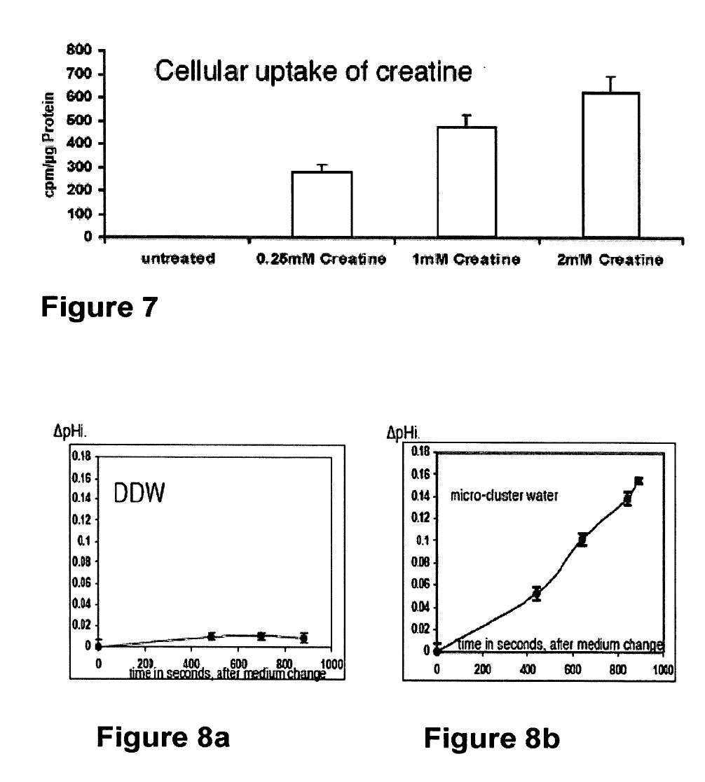

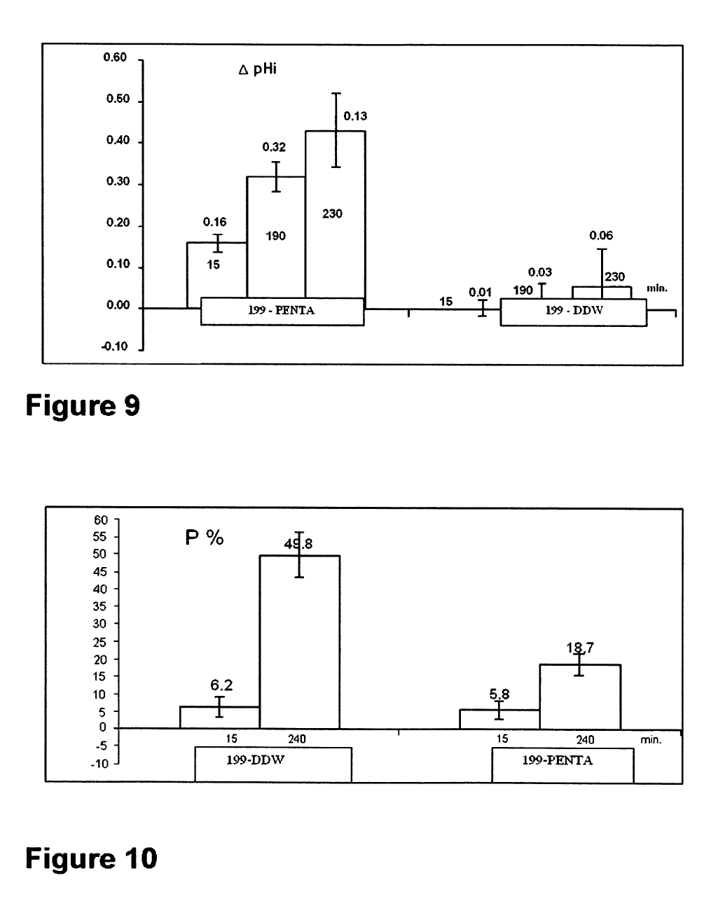

### DETAILED DESCRIPTION OF THE INVENTION

The methods of the present invention generally comprise administering to a subject an amount of a micro-cluster fluid, topically, orally, transdermally or other routes of administration known in the art. It is thought that the micro-cluster liquids modulate one or more of the ROS and/or free radicals responsible for oxidative tissue damage associated with pre-mature aging. The present invention further generally comprises incorporating neutriceutical and pharmaceutical agents in the micro-cluster fluid, together with a transdermal enhancer. It is thought that the micro-cluster liquids in association with creatine compounds modulate one or more of the structural or functional components of mtDNA mutagenesis and/or the creatine kinase/phosphocreatine system sufficient to prevent, reduce or ameliorate symptoms of aging and damage to the skin. Components of the systems which can be modulated include the rate of mtDNA mutation, intracellular pH, intercellular pH, ROS concentration, cell longevity, the enzyme creatine kinase, the substrates creatine and creatine phosphate, and the transporter of creatine. The term “modulate,” “modulation” or “modulating” includes any increase or decrease in the activity of any component of the creatine kinase/phosphocreatine system.

In one embodiment, the invention pertains to a method for treating a subject (e.g., a mammal, preferably, a human) for skin disorders by administering to the subject an effective amount of a cosmaceutical comprising a creatine compound in a micro-cluster liquid such that the skin damage is treated.

Creatine compounds are predicted to preserve tissue by boosting up energy reserves in the skin and also by arresting mechanisms involved in oxidative damage and cell death. The micro-cluster liquids are predicted to independently preserve tissue by arresting deleterious oxidative mechanisms involved in oxidative damage and cell death. The combination of creatine compounds and micro-cluster liquids provides a synergistic composition predicted to preserve tissue by boosting up energy reserves in the skin, by arresting deleterious oxidative mechanisms involved in oxidative damage and cell death and by enhanced transdermal penetration and migration of active agents. Compounds, which are particularly effective for this purpose, include micro-cluster liquids, micro-cluster water, chelated minerals, chelated vitamins, creatine, creatine phosphate, taurine, osmolytes, ectoin and analogs thereof, which are described in detail below.

The term “creatine compounds” includes creatine, creatine phosphate, and compounds, which are structurally similar to creatine or creatine phosphate, and analogs of creatine and creatine phosphate, including salts thereof such as creatine pyruvate. The term “creatine compounds” also includes compounds, which “mimic” the activity of creatine, creatine phosphate or creatine analogs. The term “mimics” is intended to include compounds, which may not be structurally similar to creatine but mimic the therapeutic activity of creatine, creatine phosphate or structurally similar compounds. Also the term creatine compound includes “modulators of the creatine kinase system,” for example, compounds which modulate the activity of the enzyme, or the activity of the transporter of creatine or the ability of other proteins or enzymes or lipids to interact with the system.

The term “treatment” includes the diminishment or alleviation of at least one symptom associated or caused by the disorder being treated. For example, treatment can be diminishment of several symptoms of a disorder or complete eradication of a disorder.

The language “treating for skin disorders” includes both prevention of disorders, amelioration and/or arrest of the disorder process. Examples of skin disorders include, but are not limited to aging and damage resulting from sun radiation, stress, fatigue and/or free radicals. Although not wishing to be bound by theory, The micro-cluster liquids in association with creatine compounds described herein are thought to have both curative and prophylactic effects on development of damage and aging of the skin and other tissue. The language also includes any amelioration or arrest of any symptoms associated with the disorder process (e.g., wrinkles). For example, treating wrinkles may include preventing, retarding, arresting, or reversing the process of wrinkle formation in skin, e.g., mammalian skin, preferably, human skin.

This invention is directed to reducing free-radical and oxidative damage secondary to free-radicals and other oxidants contacting cells, cellular components or tissues. The micro-cluster water of the present invention has free-radical and oxidant quenching properties. Contacting cells, cellular components or tissues with the present micro-cluster water results in increased cellular longevity, decreased rates of DNA mutation, improved mitochondrial efficiency, decreased mitochondrial DNA mutation, increased collagen and fibroblast growth, amelioration of wrinkles and other skin damage; and thus anti-aging. Administration of the compositions of the present invention is accomplished by several methods, including ingestion topical application such as by a cosmetic product, or any other method whereby the tissue or system in need of treatment is sufficiently contacted.

Conventional means for administering therapeutic or cosmetics agents (“active agents”) to a human or animal are usually limited to some degree by biological, chemical, and physical barriers. Examples of physical barriers are the skin and various organ membranes that must be traversed before the agent reaches a target. Chemical barriers include pH variations, lipid bi-layers, and degrading enzymes. Both biologically and chemically active agents are particularly vulnerable to such barriers.

Many active agents can be applied topically and this provides a convenient mode of administration, particularly for cosmetics agents that are typically applied to an area of the skin that is affected by a skin condition. However, the effectiveness of topical application of an active agent depends on two major factors: a) percutaneous absorption and penetration; and b) bioavailability of the penetrated active agent to the target site in the skin.

For active agents to be effectively applied topically, the agents need to penetrate the stratum corneum (the outer layer of the skin that includes layers of terminally differentiated keratinocytes) into the epidermal layers, and then be distributed and bioavailable to the target sites to provide an effect. This transdermal migration of active agents shall be termed transdermal administration.

Many cosmetics agents require routine application over an extended time, and for this reason topical application is advantageous because the administration regime is relatively simple and can be achieved with a minimum of inconvenience. However, to maximize the effectiveness of the treatment, as much of the cosmetic agent as possible needs to be absorbed into the skin and when the agent is applied topically by applying a cream or lotion to the skin it is common for least some of the active agent to be lost by rubbing off or evaporation. The inventors have discovered that generation of nanometer sized particles of the active agents when combined with micro-cluster water, decreases this rub-off problem and improves absorption of active agents.

By way of example, for the purposes of the present invention a cosmetic agent may preferably be selected from one or more of: anti-aging agents, anti-wrinkle agents, antioxidants, anti-scarring agents, phytoestrogens, isoflavones, coumarines, lip balms, free radical quenching agents, and antiseptic anti-acne agents.

As used herein, the term “cosmetic agent” means any compound, mixture of compounds, or preparations derived their from that are intended to be placed in contact with external parts or with mucosal membranes of an animal body. (Especially a human body) with a view to cleaning, changing the appearance, protecting and/or keeping the body parts to which the agent is applied in good condition.

Preferably, the cosmetics agent is capable of diminishing, reducing or preventing the effects of one or more skin conditions including: the visible effects of aging, wrinkles, acne, age spots, scars (keloids) broken capillaries and, includes compositions which also optionally cleanse the skin, preferably in the form of liquid compositions such as liquid soaps, lotions and solutions both additives and compositions for application to skin, hair, scalp, nails, eyes or teeth.

As used herein, the term “cosmaceutical” means a cosmetic agent according to the present invention, which is adapted to facilitate delivery of neutriceutical compositions comprising vitamins, minerals and osmolytes.

The term “minerals” as used herein means the inorganic compounds normally part of the class, as known in the art. Examples of metabolically important minerals are well documented in numerous health, wellness and medical texts including for example, Sb, As, B, Br, Yb, Pd, Re, F, Ir, La, W, Cs, C, Pt, Tm, N, Ni, Ta, Tb, Fe, K, I, Co, Mo, V, Ag, Mg, Cr, Cu, Zn, Ca, Si, Sn, Ni, P and S. A chelating matrix delivery system is preferably used to facilitate transdermal delivery of these minerals, as part of the present compositions. Such a chelating matrix delivery system is described in U.S. Pat. No. 6,716,458 to Tarbet, filed Aug. 7, 2000, which is incorporated herein by reference. The incorporation of these minerals in a chelating matrix,

As used herein, the term “osmolyte” means organic solutes accumulated by cells/tissues in response to osmotic stress. In general, osmolytes increase thermodynamic stability of folded proteins and provide protection against denaturing stresses. Examples of osmolytes includes, but is not limited to, creatines, taurins, ectoins, their derivatives and corresponding biologically compatible salts.

Another form of the present invention provides a method of enhancing penetration of the cosmetic and/or neutriceutic agent through the skin, the method including the steps of applying to the skin a composition containing: at least one cosmetic or neutriceutic agent, and a dermal penetration enhancer. The topical application of this composition results in the delivery of the active agents into the stratum corneum as well as delivery of the active agents into the epidermis and dermis.

Although not bound by any proposed theory, the present micro-cluster water has increased potential energy as compared with double distilled water. Perhaps because of this increased energy, the micro-cluster water of the present invention is able to quench free-radicals and function as an anti-oxidant.

The term “micro-clustered composition” as used herein refers to a composition which comprises micro-cluster water. The adjective “micro-clustered” which modifies any of the compositions of bio-affecting agents, body-treating agents, adjuvant or carriers, or ingredients thereof refers to micro-clustered water in that composition, i.e. which is dissolved in, mixed with, or otherwise combined with micro-cluster water. A micro-cluster liquid is any liquid, mixture or combination of liquids, whether or not miscible, which have been processed according to the device described and claimed in U.S. Pat. No. 6,521,248, which is incorporated herein by reference.

The interaction of water and modified water media with various biological structures and processes is mainly determined by the unique role water plays in all biological systems. Water is a major constituent in most biological processes, as well as the fluid medium through which proteins and nucleic acids interact. Apart from being known as the main medium for biological reactions, water also plays a role in determining and stabilizing hydrophilic and lipophyllic structures. Due to water's unique capabilities, it is able to influence the efficacy of various processes. However, many aspects related to the biological function of water remain unclear. There are facts, which indicate that the biological activity of water is due to a change in physical/chemical parameters. One of the important aspects in gaining an understanding of the mechanism controlling water's biological activity is to study it at the cell level. Water is highly related to the internal regulation system, including intracellular pH and cell membrane status. Macrophage response and viability is therefore a useful indicator in this analysis.

The creatine kinase/creatine phosphate energy system is only one component of an elaborate energy-generating system found in tissue with high and fluctuating energy requirements. The components of the creatine energy system include the enzyme creatine kinase, the substrates creatine and creatine phosphate, and the transporter of creatine. Some of the functions associated with this system include efficient regeneration of energy in cells with fluctuating and high energy demands, energy transport to different parts of the cell, phosphoryl transfer activity, ion transport regulation, and involvement in signal transduction pathways.

The present invention relates to methods for protecting skin tissue against age related damage or insults such as harmful UV radiation, stress and fatigue by preserving energy pools and protecting against free radical production and oxidative stress. This is achieved by administering an amount of a creatine compound or compounds together with a micro-cluster fluid, which modulates one or more of the biological pathways involved in energy and aging sufficient to prevent, reduce or ameliorate skin damage or skin aging. Compounds which are effective for this purpose include, micro-cluster liquids, such as micro-cluster water, osmolytes such as taurine and ectoin and the natural compound creatine in its different hydration or salt and analogs of and combinations thereof. The compounds can be mixed in with creams, oils, emulsions and the like to be spread readily on skin surfaces. Alternatively, the compounds also can be packaged in a supplement form for ingestion.

The present invention also provides micro-cluster liquid based compositions containing creatine compounds in combination with a pharmaceutically or cosmetically acceptable carrier, and effective amounts of other agents which act on tissue preservation such as antioxidants (e.g., CoQ10), vitamins such as C, B₅, B₆, B₉, E, energy enhancing agents (for example creatine, chelated minerals, pyruvate, nicotinamide) osmolytes and skin softeners to slow the process of aging.

The term “modulate,” “modulation” or “modulating” includes any increase or decrease in the activity of any component of an affected biological pathway or system.

Micro-cluster liquids in combination with creatine compounds are predicted to preserve tissue by boosting up energy reserves in the skin and also by arresting mechanisms involved in oxidative damage and cell death. Compounds which are particularly effective for this purpose include micro-cluster water in combination with creatine, creatine phosphate, and analogs thereof which are described in detail below. The term “creatine compounds” includes creatine, creatine phosphate, and compounds which are structurally similar to creatine or creatine phosphate, and analogs of creatine and creatine phosphate. The term “creatine compounds” also includes compounds, which “mimic” the activity of creatine, creatine phosphate or creatine analogs. The term “mimics” is intended to include compounds, which may not be structurally similar to creatine but mimic the therapeutic activity of creatine, creatine phosphate or structurally similar compounds. Also the term creatine compound includes “modulators of the creatine kinase system,” for example, compounds which modulate the activity of the enzyme, or the activity of the transporter of creatine or the ability of other proteins or enzymes or lipids to interact with the system.

The term “treatment” includes the diminishment or alleviation of at least one symptom associated or caused by the disorder being treated. For example, treatment can be diminishment of several symptoms of a disorder or complete eradication of a disorder.

The language “treating for skin disorders” includes both prevention of disorders, amelioration and/or arrest of the disorder process. Examples of skin disorders include, but are not limited to aging and damage resulting from sun radiation, stress, fatigue and/or free radicals. Although not wishing to be bound by theory, The micro-cluster liquids in association with creatine compounds described herein are thought to have both curative and prophylactic effects on development of damage and aging of the skin and other tissue. The language also includes any amelioration or arrest of any symptoms associated with the disorder process (e.g., wrinkles). For example, treating wrinkles may include preventing, retarding, arresting, or reversing the process of wrinkle formation in skin.

The term “topical administration” includes methods of delivery such as laying on or spreading on the skin. It involves any form of administration, which involves the skin. Examples of compositions suitable for topical administration include but are not limited to, ointments, lotions, creams, cosmetic formulations, and skin cleansing formulations. Additional examples include aerosols, solids (such as bar soaps) and gels.

The term “pharmaceutically acceptable” includes drugs, medicaments or inert ingredients which are suitable for use in contact with the tissues of humans and lower animals without undue toxicity, incompatibility, instability, irritation, allergic response, and the like, commensurate with a reasonable benefit/risk ratio. The term also encompasses cosmetically acceptable ingredients.

The language “therapeutically or cosmetically effective amount” is intended to include the amount of the compound sufficient to prevent onset of aging or damage to the skin or significantly reduce progression of damage in the subject being treated. A therapeutically or cosmetically effective amount can be determined on an individual basis and will be based, at least in part, on consideration of the severity of the symptoms to be treated and the activity of the specific analog selected if an analog is being used. Further, the effective amounts of the compound may vary according to the age of the subject being treated. Thus, a therapeutically or cosmetically effective amount of the compound can be determined by one of ordinary skill in the art employing such factors as described above using no more than routine experimentation in health care management.

The topical pharmaceutical compositions of the present invention may be made into a wide variety of product types. These include, but are not limited to solutions, lotions, creams, beach products, gels, sticks, sprays, pads, ointments, pastes, mousses and cosmetics. These product types may comprise several types of carrier systems including, but not limited to solutions, emulsions, gels and solids.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

The topical pharmaceutical compositions of the present invention may also be formulated as makeup products such as foundations.

The topical pharmaceutical compositions of the present invention may also be formulated as medicated pads. Suitable examples of these pads are fully disclosed in U.S. Pat. Nos. 4,891,227 and 4,891,228, to Thaman et al., both issued Jan. 2, 1990 the disclosures of which are incorporated herein.

The topical pharmaceutical compositions of the present invention may contain, in addition to the aforementioned components, a wide variety of additional oil-soluble materials and/or water-soluble materials conventionally used in topical compositions, at their art-established levels.

Various water-soluble materials may also be present in the compositions of this invention. These include humectants, proteins and polypeptides, preservatives and an alkaline agent. In addition, the topical compositions herein can contain conventional cosmetic adjuvants, such as dyes, pigments and perfumes.

The topical pharmaceutical compositions of the present invention may also include a safe and effective amount of a dermal penetration enhancing agent. A preferred amount of penetration enhancing agent is from about 1% to about 5% of the composition. Another useful penetration enhancer for the present invention is the non-ionic polymer under the CTFA designation: polyacrylamide and isoparrafin and laureth-7, available as Sepigel from Seppic Corporation. Also useful is polyquaternium-32 and mineral oil known as SalCare SC92 available from Allied Colloids, Suffolk, Va. This is a class of cationic polymers which are generally described in U.S. Pat. No. 4,628,078 to Glover et al. issued Dec. 9, 1986 and U.S. Pat. No. 4,599,379 to Flesher et al. issued Jul. 8, 1986 both of which are incorporated by reference herein.

Examples of useful penetration enhancers, among others, are disclosed in U.S. Pat. No. 4,537,776, Cooper, issued Aug. 27, 1985; U.S. Pat. No. 4,552,872, Cooper et al., issued Nov. 12, 1985; U.S. Pat. No. 4,557,934, Cooper, issued Dec. 10, 1985; U.S. Pat. No. 4,130,667, Smith, issued Dec. 19, 1978; U.S. Pat. No. 3,989,816, Rhaadhyaksha, issued Nov. 2, 1976; U.S. Pat. No. 4,017,641, DiGiulio, issued Apr. 12, 1977; and European Patent Application 0043738, Cooper et al., published Jan. 13, 1982.

Other conventional skin care product additives may also be included in the compositions of the present invention. For example, collagen, hyaluronic acid, elastin, hydrolysates, primrose oil, jojoba oil, epidermal growth factor, soybean saponins, mucopolysaccharides, and mixtures thereof may be used.

Various vitamins and minerals may also be included in the compositions of the present invention. For example, Vitamin A, ascorbic acid, Vitamin B, biotin, panthothenic acid, Vitamin D, Vitamin E and mixtures thereof and derivatives thereof are contemplated.

Also contemplated are skin cleaning compositions comprising both active compounds of the present invention and a cosmetically-acceptable surfactant. The term “cosmetically-acceptable surfactant” refers to a surfactant, which is not only an effective skin cleanser, but also can be used without undue toxicity, irritation, allergic response, and the like. Furthermore, the surfactant must be capable of being commingled with the active compound in a manner such that there is no interaction, which would substantially reduce the efficacy of the composition for regulating skin damage, e.g., wrinkles.

The skin cleaning compositions of the present invention preferably contain from about 0.1% to about 20%, preferably from about 1% to about 5%, of the creatine compound (e.g., creatine, cyclocreatine or another creatine compound) and from about 1% to about 90% micro-cluster liquid, and from about 0.1% to about 10%, of a cosmetically-acceptable surfactant.

The physical form of the skin cleansing compositions is not critical. The compositions can be, for example, formulated as toilet bars, liquids, pastes, mousses, or pads.

The cleaning compositions of the present invention can optionally contain, at their art-established levels, materials, which are conventionally used in skin cleansing compositions.

Sunblocks and sunscreens incorporating micro-cluster liquids and creatine compounds are also contemplated. The term “sun block” or “sun screen” includes compositions, which block UV light. Examples of sunblocks include, for example, zinc oxide and titanium dioxide.

Sun radiation is one major cause of skin damage, e.g., wrinkles. Thus, for purposes of wrinkle treatment or prevention, the combination of a micro-cluster liquid and a creatine compound with a UVA and/or UVB sunscreen would be advantageous. The inclusion of sunscreens in compositions of the present invention will provide immediate protection against acute UV damage. Thus, the sunscreen will prevent further skin damage caused by UV radiation, while the compounds of the invention modulates existing skin damage.

A wide variety of conventional sunscreening agents are suitable for use in combination with the active compound. Segarin, et al., at Chapter VIII, pages 189 et seq., of Cosmetics Science and Technology, disclose numerous suitable agents. Specific suitable sunscreening agents include, for example: p-aminobenzoic acid, its salts and its derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (methyl and benzyl esters, .alpha.-phenyl cinnamonitrile; butyl cinnamoyl pyruvate); Dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbotol) (6-propyl piperonyl) ether; Hydroquinone; Benzophenones (Oxybenzene, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Iso-propyldibenzoylmethane; Butylmethoxydibenzoylmethane; Etocrylene; and 4-isopropyl-di-benzoylmethane.

Preferred sunscreens useful in the compositions of the present invention are nanometer particles of TiO₂, ZnO, dispersed in a micro-cluster liquid and mixtures thereof.

A safe and effective amount of sunscreen may be used in the compositions of the present invention. The sunscreening agent must be compatible with the active compound. Generally the composition may comprise from about 1% to about 20%, preferably from about 2% to about 10%, of a sunscreening agent. Exact amounts will vary depending upon the sunscreen chosen and the desired Sun Protection Factor (SPF).

An agent may also be added to any of the compositions of the present invention to improve the skin substantivity of those compositions, particularly to enhance their resistance to being washed off by water, or rubbed off. A preferred agent, which will provide this benefit is a copolymer of ethylene and acrylic acid. Compositions comprising this copolymer are disclosed in U.S. Pat. No. 4,663,157, Brock, issued May 5, 1987, which is incorporated herein by reference.

In another embodiment of the present invention, an anti-inflammatory agent is included as an active agent along with the micro-cluster liquids in association with creatine compounds of the invention. The anti-inflammatory agent protects strongly in the UVA radiation range (though it also provides some UVB protection as well) thereby preventing further skin damage caused by UV radiation, while The micro-cluster liquids in association with creatine compounds of the invention treat existing damage. Thus the combination provides broad protection against further damage while facilitating repair of pre-existing damage. The topical use of anti-inflammatory agents reduces photo-aging of the skin resulting from chronic exposure to UV radiation. (See U.S. Pat. No. 4,847,071, Bissett, Bush, and Chatterjee, issued Jul. 11, 1989, incorporated herein by reference; and U.S. Pat. No. 4,847,069, Bissett and Chatterjee, issued Jul. 11, 1989, incorporated herein by reference.)

A safe and effective amount of an anti-inflammatory agent may be added to the compositions of the present invention, preferably from about 0.1% to about 10%, more preferably from about 0.5% to about 5%, of the composition. The exact amount of anti-inflammatory agent to be used in the compositions will depend on the particular anti-inflammatory agent utilized since such agents vary widely in potency.

In another embodiment, the cosmaceutical further comprises a safe and effective amount of a skin protectant. The skin protectant preferably comprises from about 0.001% to about 2%, more preferably from about 0.01% to about 1% of the composition. Useful skin protectants are disclosed in the Federal Register Vol. 48, No. 32 and include allantoin, aluminum hydroxide gel, bismuth subnitrate, boric acid, calamine, cocoa butter, corn starch, dimethicone, glycerin, kaolin, live yeast cell derivative, petrolatum, shark liver oil, sodium bicarbonate, sulfur, tannic acid, white petrolatum, zinc acetate, zinc carbonate and zinc oxide and mixtures thereof.

Formulations of the present invention include those suitable for topical, oral, nasal, transdermal, buccal, sublingual, rectal, vaginal and/or parenteral administration. The formulations may conveniently be presented in unit dosage form and may be prepared by any methods well known in the art of pharmacy.

Methods of preparing these formulations or compositions include the step of bringing into association all components of the formulation, including accessory ingredients. This mixture of ingredients is processed through the device as described in U.S. Pat. No. 6,521,248 until desired nanometer particle size and/or dissolution of hydrophobics is accomplished.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the present invention to the body. Such dosage forms can be made by dissolving or dispersing the compounds in the proper medium. Absorption enhancers can also be used to increase the flux of the compound across the skin. The rate of such flux can be controlled by either providing a rate controlling membrane or dispersing the active compounds in a polymer matrix or gel.

These compositions may also contain adjuvants such as preservatives, wetting agents, emulsifying agents and dispersing agents. Prevention of the action of microorganisms may be ensured by the inclusion of various antibacterial and antifingal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In a further embodiment, the skin disorder is associated with free radicals, aging, sun radiation, stress or fatigue. In another embodiment, the subject is afflicted with wrinkles or is at risk for a skin disorder.

The term “associated with free radicals” includes any disorders or damage to the skin resulting directly or indirectly from free radicals. The free radicals may be initiated by, for example, sun radiation (e.g., UV radiation) or pollution.

The term “aging” includes processes where there is oxidative damage, energy depletion or mitochondrial dysfunction where onset, amelioration, arrest, or elimination is effectuated by The micro-cluster liquids in association with creatine compounds described herein. Symptoms of aging include, but are not limited to, wrinkles, loss of elasticity of the skin and uneven pigmentation of the skin.

The invention also features a composition for the treatment of the skin of a subject. The composition comprises an effective amount of a micro-cluster liquid combined with a creatine, creatine phosphate, a creatine compound or a salt thereof. Preferably, the effective amount is effective to treat or prevent a skin disorder. Preferably, the composition is suitable for topical administration. The composition may be formulated as a lotion, cream, or ointment, gel or solid. In one advantageous embodiment, the composition also contains a sunblock or sunscreen (e.g., zinc oxide or titanium dioxide).

In another further embodiment, the composition may be formulated as a cosmetic foundation or as a skin cleansing agent. Advantageously, the composition may contain a penetration agent. Examples of compounds which may be incorporated into the composition of the invention include, but are not limited to, hydroxyacids, retinols, Aloe, Chamomile, or mixtures thereof.

In a further embodiment, the skin disorder is associated with free-radicals, aging, sun radiation, stress or fatigue.

In a further embodiment, the invention contemplates co-administering to the subject an effective amount of a skin preserving agent. Examples of skin preserving agents include antioxidants, such as micro-cluster water, ascorbic acid, vitamins, coenzyme Q10 (CoQ10) and its derivatives, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Preferred anti-oxidants include, CoQ10 and vitamin E. Other examples of skin preserving agents include energy-enhancing agents (e.g., ATP, nicotinamide or pyruvate), vitamins (e.g., E, C, B5, B6, and B9) and vitamin precursors.

The term “energy enhancing agents” also includes stimulants of mitochondrial function or ATP production elsewhere in the cell. Examples include intermediates such as, for example, pyruvate, nicotinamide and CoQ10.

Aging Oxidative Stress and Mitochondrial Dysfunction:

A common feature of the life cycle of virtually all multicellular organisms is the progressive decline in efficiency of various physiological processes once the productive phase of life is over. Data has supported the hypothesis that senescence cell death secondary to loss of functional capacity is due to accumulation of molecular oxidative damage (Harman 1956; Stadtman 1992; Ames et. al., 1993; Sohal 1995). The hypothesis is based on the fact that oxygen is potentially a toxic substance, and its use by aerobes, although necessary for their immediate survival, also may be hazardous to their long term existence. Molecular oxygen is the precursor of superoxide, hydrogen peroxide and hydroxyl radicals. Upon further reactions these could generate reactive oxygen species that cause extensive oxidative damage to macromolecules. Lipid peroxidation, DNA damage and carbonylation of proteins are some of the devastating effects. During aging there is an increase in the amount of oxidative stress which could be a result of increase in the rate of generation of reactive oxygen species, or the decline in anti-oxidative defenses or the decline in the efficiency of repair or removal of damaged molecules (Sohal et. al., 1996). With aging there is an increase in the production of ROS (Reactive Oxygen Species) from mitochondria which results in damage to the inner mitochondrial membrane. By positive feedback mechanisms this results in further increase in ROS. Among flies, those with a longer life expectancy were shown to exhibit a lower rate of mitochondrial superoxide, hydrogen peroxide generation, a lower rate of protein oxidative damage, less DNA oxidative damage, higher activities of SOD and catalase, increased glutathione a versatile intracellular reductant. Variations in maximum life span among different species are often associated with differences in the metabolic rate (rate of oxygen consumption), metabolic potential (total amount of energy consumed per gram of body weight during life span) and level of oxidative stress. The highest degree of oxidative damage occur in tissues such as brain, heart and skeletal muscle which are composed primarily of long lived postmitotic cells. These tissues are also the targets of several age related degenerative disorders in which oxidative stress has been implicated (Davies 1995; Weindruch et al., 1993). Agents that minimize the production of reactive oxygen species are predicted to be protective.

Creatine Kinase Skin Aging and Skin Damage

The creatine content and the efficiency of the creatine kinase system decreases with aging. Aging and several insults result in oxidative stress state and energy compromise. Minimizing the rate of production of molecules associated with oxidative damage correlates well with a decrease in oxidative damage. Such minimization combined with energy boosting effects should slow damage to tissue during aging or exposure to insults. Creatine and analogs of creatine that modify the rate of ATP synthesis through creatine kinase could sustain energy production, mitochondrial function, and protect against free radical production. Such effects could have positive impact against aging or insult related skin damage.

Without wishing to be bound by theory, it is thought that modulating the creatine kinase activity would modulate energy flow and affect skin cell function, integrity and survival. An activated energy state should minimizes oxidative damage and enable cells to withstand insult secondary to aging or insults such as UV radiation.

Creatine is taken by athletes to boost muscle function during burst activity (for review see Wyss and Kaddurah-Daouk 1999) and during competitions. Creatine was shown to have neuroprotective properties in several animal models of neurodegenerative diseases (Matthews et al., 1988; Kliveny et al 1999; Matthews et. al., 1999).

Ingestion of creatine analogs has been shown to result in replacement of tissue phosphocreatine pools by synthetic phosphagens with different kinetic and thermodynamic properties. This results in subtle changes of intracellular energy metabolism, including the increase of total reserves of high energy phosphate (see refs. Roberts, J. J. and J. B. Walker, Arch Biochem. Biophys 220(2): 563-571 (1983)). The replacement of phosphocreatine pools with slower acting synthetic phosphagens, such as creatine analogs might benefit neurological disorders by providing a longer lasting source of energy. One such analog, cyclocreatine (1-carboxymethyl-2-aminoimidazolidine) modifies the flow of energy of cells in stress and may interfere with ATP utilization at sites of cellular work.

Similarly, ingestion of micro-cluster water has been shown to improve cellular energy metabolism. Moreover, the topical application of micro-cluster water has been shown to provide a significant decrease in mtDNA mutation rates.

Creatine Compounds Useful in Skin Care

Creatine compounds useful in the present invention include compounds which modulate one or more of the structural or functional components of the creatine kinase/phosphocreatine system. Compounds which are effective for this purpose include creatine, creatine phosphate and analogs thereof, compounds which mimic their activity, and salts of these compounds as defined above. Exemplary creatine compounds are described below.

Creatine (also known as N-(aminoiminomethyl)-N-methylglycine; methylglycosamine or N-methyl-guanido acetic acid) is a well-known substance. (See, The Merck Index, Eleventh Edition, No. 2570 (1989).

Cyclocreatine is an essentially planar cyclic analog of creatine. Although cyclocreatine is structurally similar to creatine, the two compounds are distinguishable both kinetically and thermodynamically. Cyclocreatine is phosphorylated efficiently by creatine kinase in the forward reaction both in vitro and in vivo. Rowley, G. L., J. Am. Chem. Soc. 93: 5542-5551 (1971); McLaughlin, A. C. et. al., J. Biol. Chem. 247, 4382-4388 (1972).

The phosphorylated compound phosphocyclocreatine is structurally similar to phosphocreatine; however, the phosphorous-nitrogen (P—N) bond of cyclocreatine phosphate is more stable than that of phosphocreatine. LoPresti, P. and M. Cohn, Biochem. Biophys. Acta 998: 317-320 (1989); Annesley, T. M. and J. B. Walker, J. Biol. Chem. 253; 8120-8125, (1978); Annesley, T. M. and J. B. Walker, Biochem. Biophys. Res. Commun. 74:185-190 (1977).

Guanidino acetate is yet another analog of creatine and is a precursor of creatine in its biosynthetic pathway. Guanidino benzoic acids are structurally related to creatine. Also compounds that attach amino acid like molecules covalently to creatine are creatine compounds of interest. Examples are creatine-ascorbate and creatine-pyruvate. Other types of molecules could be covalently attached.

Creatine analogs and other agents which act to interfere with the activity of creatine biosynthetic enzymes or with the creatine transporter are useful in the present method of treating or preventing age related damage. Thus the effects of such compounds can be direct or indirect, operating by mechanisms including, but not limited to, influencing the uptake or biosynthesis of creatine, the function of the creatine phosphate shuttle, enzyme activity, or the activity of associated enzymes, or altering the levels of substrates or products of a reaction to alter the velocity of the reaction.

Compounds which modify the structure or function of the creatine kinase/creatine phosphate system directly or indirectly are useful in preventing and/or treating age related damage to tissue such as skin.

Molecules that regulate the transporter of creatine, or the association of creatine kinase with other protein or lipid molecules in the membrane, the substrates concentration creatine and creatine phosphate also are useful in preventing and/or treating age related damage to tissue such as skin.

Compounds which are useful in the present invention can be substrates, enzyme activity modifiers or substrate analogs of creatine kinase. In addition, modulators of the enzymes that work in conjunction with creatine kinase now can be designed and used, individually, in combination or in addition to creatine compounds. Combinations of creatine compounds with other supplements or other drugs is proposed.

The pathways of biosynthesis and metabolism of creatine and creatine phosphate can be targeted in selecting and designing compounds which may modify energy production or high energy phosphoryl transfer through the creatine kinase system. Compounds targeted to specific steps may rely on structural analogies with either creatine or its precursors. Novel creatine analogs differing from creatine by substitution, chain extension, and/or cyclization may be designed. The substrates of multisubstrate enzymes may be covalently linked, or analogs which mimic portions of the different substrates may be designed. Non-hydrolyzable phosphorylated analogs can also be designed to mimic creatine phosphate without sustaining ATP production.

Creatine, creatine phosphate and many creatine analogs are commercially available. Additionally, analogs of creatine may be synthesized using conventional techniques.

Creatine compounds which currently are available or have been synthesized include, for example, creatine, b-guanidinopropionic acid, guanidinoacetic acid, creatine phosphate disodium salt, cyclocreatine, homocyclocreatine, phosphinic creatine, homocreatine, ethylcreatine, cyclocreatine phosphate dilithium salt and guanidinoacetic acid phosphate disodium salt, 4 guanidino benzoic acid and derivatives, creatine-pyruvate, creatine-ascorbate among others.

The term “administration” is intended to include routes of administration which allow the inventive compositions to perform their intended function(s).

### EXAMPLES

Numerous types of non-mineralized drinking waters have potential mutagenic effects because of various free radical components that may be present secondary to a multi-step water purification and processing system. The cytogenetic method represents one approach for evaluating the potential mutagenic effects of water. This approach is based on determination of the frequency of chromosome aberrations, sister chromatid exchange (SCE), and cell cycle duration. The method was used in testing the cytogenetic effects of Micro-cluster research water having the trade name AQUA RX™ in the U.S. market. This water was provided by Bio-Hydration Research Lab (USA). Micro-cluster water is produced through a multistep process according to U.S. Pat. No. 6,521,248 which provides research water with unique attributes and a purity of less than 0.5 ppm of total dissolved substances (TDS). Medicinal grade oxygen is added to the water in a final step to pressurize the plastic bottles for shipment. Cell culture medium was prepared by dissolving RPMI 1640 (Gibco) powder in standard deionized water—18 Mohm (control) or Micro-cluster water. Experiments were conducted with human lymphocytes, which were cultured in accordance with standard protocol. Cells are fixed after 48 hours of culturing in order to determine the frequency of chromosome aberrations. Upon determining the SCE frequency, after 48 hours 5-BDU (10 mg/ml) is added to the cell culture. Cells are fixed after 80 hours of culturing. Specimen preparation and staining are done according to procedures known in the art. Experiments were performed twice for each of the 3 donors. 1200 metaphases are analyzed to determine the chromosome aberrations in the control and in Micro-cluster water.

### Example 1

### Effects of 1% Creatine Supplementation on 3-Nitrotyrosine/Tyrosine Concentration in FALS Mice

Oxidative injury involves the activation of nitric oxide production, and peroxynitrite which results in nitration of proteins. The nitration of proteins could be determined by measuring the ratio of 3-nitrotryrosine to tyrosine. The FALS mice are transgenic animals that express a mutant form of Cu/Zn superoxide dismutase found in patients with familial ALS (Amyotrophic Lateral Sclerosis). These animals develop ALS symptoms with gradual motor neuron loss, muscle weakness, and die within 135 days. Oxidative stress has been associated with the death of motor neurons. Levels of 3-nitrotyrosine are significantly increased in the spinal cords of these mice (Ferrante 1997). The transgenic mice with the G93A mutation and the littermate controls (eight mice per group) were fed 1% creatine or unsupplemented diets at days 70 of age and then killed at 120 days of age for measurements of 3-nitrotyrosine as described (Ferrante 1997). Creatine ingestion can significantly inhibit the higher levels of 3 nitrotyrosine/tyrosine levels in lower spinal cords of transgenic FALS mice.

### Example 2

### Effect of 1% Creatine Supplementation on Hydroxyl Radical Production as Measured by Rate of Conversion of Salicylate to its by Products in FALS Mice

The level of free radical production in vivo can be determined using the microdialysis technique (Matthews et al 1998). Administration of the mitochondrial toxin 3-nitropropionic acid results in a significant increase in the conversion of salicylate to 2,3-DHBA in the striatum, which is blocked in mice over expressing Cu, Zn SOD (Bogdanov et. al., 1998). Here we demonstrate that systemic administration of 3-nitropropionic acid (3-NP) resulted in a significant increase in the conversion of 4-HBA to 3,4-DHBA in G93 A transgenic mice fed unsupplemented diets. In animals fed 1% creatine supplemented diets, there was no significant increase in 3,4 DHBA/4HBA after 3-NP administration. This demonstrates that creatine can minimize the production of hydroxyl radicals that are implicated in aging related damage.

### Example 3

### Production of 2,3 and 2,5 DHBA and 3 Nitrotyrosine (Indicators of Oxidative Stress) after Intrastriatal Injection of Malonate in Control Animals Fed with Creatine and Those Fed with Cyclocreatine

The salicylate hydroxyl radical-trapping method was used for measuring levels of hydroxyl radicals in striatal tissue after malonate injections. Eight animals in each group were fed either a normal diet or a diet enriched with 1% creatine or 1% cyclocreatine for two weeks before intrastriatal malonate injections. Animals were injected with 200 mg/kg salicylate intraperitoneally just before the malonate injections and were killed 1 hour later. The striata were then dissected rapidly from a 2-mm thick slice and placed in 0.25 ml of chilled 0.1 M perchloric acid. Samples were subsequently sonicated, frozen rapidly and thawed and centrifuged twice. An aliquot of supernatant was analyzed by HPLC with the 16-electrode electrochemical detection (Beal et. al., 1990). Salicylate, 2,3 and 2,5 DHBA, tyrosine, 3-nitrotyrosine were measured electrochemically by oxidation at 840, 240, 120, 600 and 840 mV respectively with retention times of 20.5, 9.4, 6.3, 10.5, 18.2 min respectively. The data were expressed as the ratio of 2,3 and 2,5 DHBA to salicylate to normalize the DHBA concentrations for differing brain concentrations of salicylate. Similarly, 3-nitrotyrosine levels were normalized to tyrosine levels. We also examined the effects of 1% creatine supplementation for 2 weeks on 3-NP induced increases in 3-nitrotyrosine levels. Male Sprague Dawley rats were treated with 3-NP at a dose of 20 mg/kg intraperotoneally and then killed at 3 hours. Ten animals were examined in each group. The striata were dissected and placed in chilled 0.1 M perchloric acid. 3-Nitrotyrosine and tyrosine concentrations were measured by HPLC with electrochemical detection (Matthews 1998). Statistical comparisons were made by unpaired Student's t test or by one way ANOVA followed by Fisher's protected least significant difference test for post hoc comparisons.

These Examples demonstrate that both creatine and cyclocreatine can protect against increases in levels of salisylate derivatives 2,3 DHBA and 2,5 DHBA after injection of the mitochondrial toxin malonate. This confirms that creatine compounds can indeed protect against production of hydroxyl radicals implicated in oxidative stress and mitochondrial dysfunction. These Examples further demonstrate that creatine and cyclocreatine have protective effects against nitration of proteins induced by the mitochondrial toxin malonate. Production of nitric oxide and peroxynitrite are part of the cascade of oxidative damage.

### Example 4

The frequency of aberrant metaphases is effectively lower for MICRO-CLUSTER (0.92%) in comparison with standard deionized water (2.50%) (df=1; x2=8,96; P=0.0028). To evaluate the average number of SCE per cell, 300 metaphases are analyzed in each case. It was shown that the SCE number is lower (3.38±0.120) compared to standard deionized water (4,01±0,145) (df=598; t=3,311; P=0,000985) when MICRO-CLUSTER water is used as a solvent for RPMI 1640 dry medium. Chromosome staining is also performed to determine the cell cycle duration by counting the number of 1 st, 2nd, and 3rd mitoses. The average number of divisions in cell culture is determined using the formula: (sigma □ni/2ⁱ⁻¹)/(sigma ni/2ⁱ⁻¹), where i=mitosis number, and ni=cell number of i-mitosis after 32 hours in presence of BDU. The duration of the cell cycle is calculated as 32 hours divided by the average number of divisions. The cell cycle duration is 21.2 hours for both types of water, which is in agreement with literature data.

Thus, micro-cluster water doesn't result in mutagenic effects compared to standard deionized water. Based on the data obtained, it appears that Micro-cluster water has a stabilizing effect, which results in both a lower frequency of sister chromatid exchanges and chromosome aberrations compared to standard deionized water.

### Example 5

This example examines the influence of Micro-cluster water on intracellular pH of mice peritoneal macrophages and to also assess the cell membrane status under short and long term exposure to the water.

Procedure: Determine intracellular pH of macrophages after 15 and 240 minutes of incubation time in standard lab medium prepared with both double distilled and Micro-cluster water. Study the kinetics of pHi values during first 15 minutes in standard lab medium prepared using either double distilled or Micro-cluster waters and estimate the number of cells with damaged plasma membranes in macrophage population after 15 and 240 minutes of incubation time.

Reagents:

Fluoresceindiacetate (FscDA, Sigma)

Ethidium bromide (EthBr, Sigma)

Hanks balanced salt solution

standard lab Powder medium (standard lab media)

HEPES (Sigma)

Double Distilled Water (DDW)

Micro-cluster Water

Nigericin (Sigma)

Mouse Peritoneal Macrophages.

Mice were sacrificed for macrophages isolation. Hanks solution 2 ml (10 mM HEPES, pH 7.2) had been injected into peritoneum. After the injection, the liquid enriched with macrophages was collected from the peritoneum. By double staining with EthBr and FSCDA, the integrity of the plasma membrane in the collected cells was controlled. Hanks solution was used to achieve a final cell concentration of 10⁶ cells/ml in suspension. Small amounts of cell suspension (20 ml) were placed on the glass cover slips, incubated for 45 minutes in the wet chamber and then washed with Hanks solution for removal of the cells attached to the glass surface. This was the method used for the entire experiment, in regards to cell suspension and cover slips.

Taking Counts of Cells with Damaged Membranes:

A double staining procedure with EthBr (5 mg/ml) and FSCDA (5 mg/ml) was used to count cells with damaged cell membranes. The method is based on the ability of EthBr to enter the cells with damaged membranes and bind with DNA. EthBr has a bright red fluorescence when bound to DNA. FSCDA easily penetrates into the cells from the medium and is structurally transformed to fluorescein with bright green fluorescence. Therefore, intact cells accumulate fluorescein and easily leave the cells with damaged cell membranes. As a result of this double staining, one can see intact cells with green fluorescence and red fluorescent cells with damaged cell membranes within 5 minutes of incubation time with the dyes.

Intracellular pH Measurements.

Macrophage intracellular pH measurements were done based on the microspectrofluorimetric method and using a fluorescent microscope of type LUMAM I3 (LOMO, Russia). This particular microscope has a modified system of fluorescence excitation and emission. Fluorescence excitation was performed by using a blue (λmax=435 nm) photodiode. Fluorescence was measured simultaneously at two different; λ=520 nm and λ=567 nm interference filters respectively. Fluorescence excitation and synchronous emission measurements were done using a built-in microcontroller (LA-70M4). Macrophages were incubated with pH indicator FSCDA dye (5 mg/ml) for 15 minutes. Free dye was washed out of the medium after the incubation period. Microscopic measurements were accomplished by using the water immersion objective (×40). A pH calibration curve was used in order to determine the value of intracellular pH. The calibration curve was represented as a ratio K=(I520/I570) (where I520, I570—fluorescence intensity at 520 nm and 570 nm respectively) depending upon macrophage intracellular pHi. To obtain different pHi values, macrophages were incubated in 140 MM KCl; 1 MM CaCl₂; 0.5 MM MgCl₂; and 20 MM HEPES medium. Intracellular pH values have varied within a range of 6.8 to 7.6. Intracellular pH has been adjusted to the pH of the medium by adding the ionophore antibiotic nigericine (Sigma) 5 mg/ml, which has the ability to exchange OH— for H+ in just 3 minutes. Nigericine has a high affinity for K⁺. This property allows it to stabilize to the following transmembrane equilibrium: Where i and o designate internal and external concentrations respectively. Hence intracellular pH will be close to the extracellular pH in the medium, having the same K⁺ concentration outside and inside the cells.

Incubation media: Hanks solution (10 MM Hepes) pH 7.2;

standard lab medium prepared on double distilled water with 10 MM Hepes pH 7.2;

standard lab medium prepared on Micro-cluster water with 10 MM Hepes pH 7.2;

Series 1.

Cells were incubated for 15 minutes in media containing EthBr and FSCDA. They were thoroughly washed in the extracellular media. Cell media was replaced by standard lab medium prepared either on double distilled water or on Micro-cluster water. A dead cell count was produced and observed over 30 times under the microscope and experiments with both types of medium replacement were repeated three times. Cells were incubated with FSCDA dye for 15 minutes and then washed away from the free dye in the surrounding cell medium. Cell medium was exchanged either on standard lab medium prepared on double distilled or on Micro-cluster water. Kinetics measurements of intracellular pH was done with at least 30 microscopic observations which were repeated a total of three times.

Series 2.

Cells were incubated for 230 minutes in standard lab cell media prepared with either double distilled or Micro-cluster water. Cells were consequently stained with FSCDA dye for intracellular pH and EthBr dye for dead cells. Quantification measurements were performed in a similar way, as described above and as known in the art.

Intracellular pH (delta pHi) change in macrophages after 15 minutes, 190 minutes, and 230 minutes of incubation in standard lab-cell medium prepared using Micro-cluster or double distilled water. Macrophage incubation in cell media prepared using Micro-cluster water for 230 minutes resulted in a 0.43 increase in pHi. A statistically insignificant increase in intracellular pH of macrophages was also observed when medium prepared with double distilled water was used.

The increase in pH is the result of quenching metobolic oxidants. The quenching of a hydroxyl radical would result in formation of hydroxide, causing an increase in pH. Likewise, the quenching of other free-radicals and oxidants results in chemical species which increase the pH. Importantly, the reduction of these free-radicals and oxidants, prevents their deleterious interaction with mitochondria or other biological components. This results in decreased mutation to mitochondria DNA and other DNA and cell membranes. This reduction in free-radicals, oxidants and the damage they cause provides increased health, blood oxygen levels and more efficient oxygen use. Also, the decreased rate of mitochondrial decay and destruction results in increased energy secondary to increased population of healthy mitochondria.

In general then, increasing the blood concentration of micro-cluster water results in decreased oxidative damage. Indeed, the inventive water provides anti-aging results through decreased damage to mitochondrial DNA, and increased efficiency in related energetics.

The conclusion of the experimental results discussed above confirms the importance of the present micro-cluster liquids in association with cosmaceutical compounds in protecting against cascades of oxidative stress. The process of aging is believed to involve mitochondrial dysfunction and oxidative damage resulting from the production of molecules like hydroxyl radicals, nitric oxide and peroxynitrite. Our results strongly suggest that micro-cluster liquids could indeed affect the process of aging and are therefore properly labeled as anti-aging.

Although the present invention has been described herein with reference to particular means, materials, and embodiments, the present invention is not intended to be limited to the particulars disclosed herein; rather, the present invention extends to all functionally equivalent structures, methods and uses, such as are within the scope of the appended claims.

What is claimed is:

1. A cosmaceutical comprising: an anti-oxidant, an energy providing agent, a restorative agent and a delivery agent, wherein; the anti-oxidant comprises a micro-cluster liquid, and the energy providing agent comprises an osmolyte.

2. The cosmaceutical according to claim 1 wherein the restorative agent is a vitamin.

3. The cosmaceutical according to claim 1 wherein the restorative agent is a mineral in a chelating matrix.

4. The cosmaceutical according to claim 1 wherein the delivery agent is a dermal permeation enhancer.

5. The cosmaceutical according to claim 1 wherein the osmolyte is a creatine compound.

6. The cosmaceutical according to claim 1 further comprising a sunscreen agent.

7. The cosmaceutical according to claim 1 wherein the restorative agent promotes collagen proliferation.

8. The cosmaceutical according to claim 1 wherein the anti-oxidant is a free-radical quenching agent.

9. The cosmaceutical according to claim 1 wherein the anti-oxidant is an ROS quenching agent.

10. A method for treating skin damage comprising administering an effective amount of an cosmaceutical comprising: an anti-oxidant, an energy providing agent, a restorative agent and a delivery agent, wherein; the anti-oxidant is comprised of a micro-cluster liquid, and the energy providing agent is comprised of an osmolyte.

11. The method according to claim 10 wherein the skin damage is selected from the group comprising: psoriasis, aging, un-even pigmentation, photo-induced damage, infection, hair loss, sunburn, dryness, wrinkles and physical damage.

12. The method according to claim 10 whereby mitochondrial energy generation is improved.

13. A composition comprising: a particleized sunscreen agent and a micro-cluster liquid.

14. The composition according to claim 13 wherein the particleized sunscreen agent is an inorganic sunscreen agent selected from the group comprising: TiO₂, ZnO and mixtures thereof and where the micro-cluster liquid is an anti-aging agent.

15. The composition according to claim 13 further comprising an osmolyte.

16. The composition according to claim 14 wherein the anti-aging agent is an organic compound and the micro-cluster liquid is micro-cluster water.

17. The composition according to claim 16 wherein the organic compound is selected from the group comprising: hydroxy acids, fullerines, collagen, collagen growth promoters and creatines.

18. A cosmaceutical comprising a micro-cluster liquid.

19. The cosmaceutical according to claim 18 wherein the micro-cluster liquid is micro-cluster water.

20. The cosmaceutical according to claim 19 wherein the micro-cluster water is anti-aging.
